(19) United States
(12) Patent Application Publication
(10) Pub. No.: US 2024/0353414 A1
(43) Pub. Date: Oct. 24, 2024

(54) ENGINEERED FLUORESCENT SPONTANEOUS ISOMERIZATION RATE BIOSENSORS

(71) Applicant: Calico Life Sciences LLC, South San Francisco, CA (US)

(72) Inventor: Maria Ingaramo, South San Francisco, CA (US)

(21) Appl. No.: 18/684,651

(22) PCT Filed: Aug. 18, 2022
(86) PCT No.: PCT/US2022/075162

§ 371 Date: Feb. 16, 2024

Related U.S. Application Data
(60) Provisional application No. 63/289,513, filed on Dec. 14, 2021.
(60) Provisional application No. 63/234,943, filed on Aug. 19, 2021.

(51) Int. Cl. G01N 33/68; G01N 33/58

(52) U.S. Cl. CPC G01N 33/68; G01N 33/582

(57) ABSTRACT

Described herein are countdown biosensors, and methods of using the same, comprising fluorophores which can spontaneously photoswitch between two or more states with different fluorescent properties (e.g. fluorescent intensity or fluorescent color). The countdown sensor comprises a fluorescent domain which can spontaneously photoswitch, and a sensing domain which responds to the desired input. The countdown sensor is “read” by measuring the photoswitching rate. In certain embodiments, the decay of fluorescent intensity over time (due to spontaneous photoswitching of different fluorescent domains) can be made to depend on the concentration of different small molecules, such as hydrogen ion.

### CROSS-REFERENCE TO RELATED APPLICATIONS

The present application claims the benefit of and priority to U.S. Provisional Application No. 63/234,943, filed Aug. 19, 2021, and U.S. Provisional Application No. 63/289,513, filed Dec. 13, 2021, the entire contents of which are hereby incorporated by reference in their entireties for all purposes.

### BACKGROUND

Fluorescent tags (e.g. Alexa 488 or Green Fluorescent Protein) are often used to reveal the presence, distribution, and dynamics of large (multi-kilodalton) cellular components (e.g. proteins like actin or histones). Many molecules (e.g. glucose or calcium) are too small to be tagged via traditional fluorophores, and are sometimes referred to as the “dark matter” of fluorescence microscopy. “Small-molecule” concentrations and dynamics are just as interesting to biologists as “large-molecule” dynamics, but are often neglected, since they're so much harder to measure.

A subclass of fluorescent tags exists which reveals small molecule dynamics; we'll refer to them as “fluorescent biosensors”. For example, GCaMP combines a fluorescent domain (similar to GFP) with a calcium-binding domain (similar to calmodulin). The fluorescent intensity of a GCaMP molecule changes in response to the local concentration of calcium, revealing clues about calcium concentration and dynamics. There are many other examples of similar biosensors (e.g., Perceval or Peredox) which sense other small molecules (e.g., adenosine phosphates or nicotinamides).

Many fluorescent biosensors (which we'll call “intensity sensors”) suffer from an ambiguity: if they emit more (or less) photons, it could be due to changes in the concentration of their target, but it could also be due to changes in the concentration of the fluorescent biosensor (or changes in the instrument). For some questions (e.g., did this neuron fire?), this ambiguity is irrelevant. For other questions (e.g., is the pH in this yeast vacuole higher or lower today than it was yesterday?), this ambiguity is crippling.

A small subclass of fluorescent biosensors exists (which we refer to as “lifetime sensors”) which do not suffer from this ambiguity. Lifetime biosensors reveal changes in the concentration of their small-molecule target by changing their “fluorescent lifetime” (the number of nanoseconds that typically elapses between absorption and emission of light by the fluorescent domain). Since fluorescent lifetime does not depend on the concentration of the fluorescent biosensor (or aspects of the instrument), lifetime sensors enable long-term quantitative measurement of small molecule concentrations.

Unfortunately, measuring fluorescent lifetime is much slower, more complicated, and expensive vs. measuring fluorescent intensity, forcing biologists to choose between a fast, cheap, ambiguous measurement, and a robust, quantitative, slow, expensive measurement. It's also extremely difficult and laborious to construct novel fluorescent biosensors. Conceptually simple operations (e.g. changing the color of the fluorescent domain, or the target of the binding domain) typically require several years of mutation and screening, and often end in failure.

### SUMMARY

In certain aspects, described herein are protein biosensors, comprising a fluorescent domain; and an analyte binding domain; wherein the fluorescent domain can spontaneously photoswitch by cis-trans isomerization or protonation; and wherein the rate of isomerization or rate of protonation is altered by binding of the analyte binding domain to an analyte of interest. In certain embodiments, the photoswitching changes fluorescent intensity or the fluorescent color of the fluorescent domain. In certain embodiments, the analyte binding domain is attached to the N-terminus of the fluorescent domain. In certain embodiments, the analyte binding domain is attached to the C-terminus of the fluorescent domain. In certain embodiments, the fluorescent domain is green fluorescent protein or rsCherry. In certain embodiments, the analyte of interest is hydrogen ion. In certain embodiments, the analyte of interest is hydroxy ions. In certain embodiments, pH is measured. In certain embodiments, the protein biosensor comprises SEQ ID NO. 1.

In certain aspects, described herein are methods of making a protein biosensor, comprising attaching an analyte binding domain to a fluorescent domain; wherein the fluorescent domain can spontaneously photoswitch by cis-trans isomerization or protonation; and wherein the rate of isomerization or rate of protonation is altered by binding of the analyte binding domain to an analyte of interest. In certain embodiments, the protein biosensor comprises SEQ ID NO. 1.

In certain aspects, described herein are methods of identifying the concentration of an analyte of interest in a sample, comprising contacting the sample with a protein biosensor of any one of the above claims. In certain embodiments, the change in fluorescent intensity of the biosensor is correlated with the concentration of the analyte of interest in the sample. In certain embodiments, the change of fluorescent intensity of the biosensor is not dependent on the concentration of the biosensor. In certain embodiments, the protein biosensor comprises SEQ ID NO. 1.

In certain aspects, described herein are kits comprising the protein biosensor and instructions for use.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 depicts data from a pH countdown sensor described herein in worms, and shows that relaxation sensing quantitatively reveals pH in a living worm despite substantial autofluorescent background. Main panel: With Colormap type: Hue indicates pH selected, the image luminance shows the density of sensor. Regions with no sensor appear black. Inset boxes on lower right: Colorbar; each box shows how a given pH will appear in the image's colormap. Controls: With Measurement type: Sensor relaxation rate selected, the “relaxation ratio” is used to infer pH. With Measurement type: Sensor activation rate selected, the “activation nonlinearity” is used to infer pH.

FIG. 2 depicts data from a pH countdown sensor described herein under chicken skin, and shows that the pH-Countdown, a photoswitchable fluorescent protein-based relaxation sensor quantitatively measures pH despite autofluorescent background and attenuation due to absorption and scattering. Upperleft panel: Time lapse photography of fluorescence (green) and reflected light (grayscale) during photoactivation and relaxation of pH-Countdown, in five capillary tubes, each buffered at a different pH. For scale, the capillary inner diameter is 580 μm. Lower panel: Normalized fluorescence signal vs. time for each of the capillary tubes shown in the upper panels. Filled circles are from the highlighted regions in the upper left panel, open circles are from the upper right panel. Cyan box shows photoactivation interval (470±20 nm, 10 mW/cm2). Upper right panel: Like the upper left panel, except the capillary tubes are underneath ˜1 mm of chicken skin.

FIG. 3 depicts a pH-Countdown relaxation sensor photoswitches in a live worm, but autofluorescence does not. Main panel: Raw timelapse animation (grayscale) of the middle portion of a C. elegans carrying a Peft-3::pH-Countdown::unc-54 plasmid, measured in the GFP channel of a simple fluorescence microscope. The field of view is 260×210 μm2. Solid green box marks a region containing mostly sensor, dotted magenta box marks a region containing mostly autofluorescence. Inset: Average fluorescent signal vs. time for the mostly-sensor (solid green) and mostly-autofluorescence (dotted magenta) regions marked in the main panel. Violet vertical lines show photo deactivation vs. time via violet illumination (395±12.5 nm), cyan vertical lines show photoactivation and fluorescence excitation vs. time via cyan illumination (470±12 nm, 1.7 W/cm2).

FIG. 4 depicts relaxation sensing inherently splits a single spectral channel into dynamic “signal” and static “background”. Green overlay: We define the ‘sensor’ channel as the difference between the brightest frame and the dimmest frame of the time lapse shown in FIG. 4 (i.e. the last frame minus the second frame). Magenta overlay: We define the ‘background’ channel as a linear combination of the brightest frame and the dimmest frame of the time lapse shown in FIG. 4 (i.e. 1.53 times the second frame minus 0.53 times the last frame).

FIG. 5 depicts distinct tagged structures in C. elegans photoactivate and spontaneously relax at very different rates. Main panel: Background-subtracted animation (green colormap) of the time lapse from FIG. 4, now including relaxation after photoactivation. Dotted blue box marks a low-pH region with slow photoactivation and relaxation rates, solid yellow box marks a high-pH region with rapid photoactivation and relaxation rates. Inset: Average fluorescent signal vs. time for the low-pH (dotted blue) and high-pH (solid yellow) regions marked in the main panel. Violet vertical lines show photo deactivation vs. time via violet illumination (395±12.5 nm), cyan vertical lines show photo activation and fluorescence excitation vs. time via cyan illumination (470±12 nm, 1.7 W/cm²), black horizontal bar shows the illumination-free relaxation interval.

FIG. 6 depicts relaxation sensing noninvasively reveals quantitative organelle-specific drug-induced pH dynamics in a highly opaque living mouse. Left panel: Raw time lapse animation (grayscale) of the back of a BALB/cJ mouse carrying a subcutaneous tumor expressing mito-pH-Countdown, measured in the GFP channel of a simple fluorescence photography system. The field of view is TODO: Maria. Orange/blue/green box marks a region expressing mito-pH-Countdown. Right panel: Average fluorescent signal vs. time for two measurements before(orange, dots) and two measurements after (blue or green, triangles) administration of the mitochondrial uncoupler BAM15. Cyan box shows photoactivation interval (470±20 nm, 60 mW/cm2). Light gray lines show calibration measurements for pH 7.5 (solid) and pH 8 (dashed).

### DETAILED DESCRIPTION

Disclosed herein are relaxation sensors that are quantitative, robust against sample opacity and autofluorescence, and compatible with simple time lapse imaging systems. A fundamental strength of relaxation sensing is that spontaneous decay signals do not resemble other signals that biological samples typically produce. To enable genetically-expressed relaxation sensors, Countdown was engineered, which is a photo switchable fluorescent protein which rapidly spontaneously equilibrates (“relaxes”) to a nonfluorescent state. To demonstrate relaxation sensing, pH-Countdown was further engineered, a relaxation sensor with rapid pH-dependent relaxation rates. pH-Countdown quantitatively reports pH in living organisms such as yeast, worms, and mice, despite substantial autofluorescence and opacity.

### Definitions

Terms used in the claims and specification are defined as set forth below unless otherwise specified.

As used herein, the term “biosensor” refers to a molecule that can detect a desired biological phenomenon or characteristic of interest.

As used herein, the term “pH biosensor” refers to a molecule that can be used to detect pH in cells and animals in vivo.

As used herein, the term, “analyte binding domain” or “sensor domain” refers to a protein domain that can bind to an analyte (e.g., molecule or ion) of interest.

As used herein, the term “isomerization rate” or “photoswitching rate” refers to the rate of cis-trans isomerization or protonation of a fluorophore.

As used herein, the term “spontaneous isomerization rate sensors”, “Countdown”, “Countdown sensor” or “Countdown biosensor” refers to a protein comprising the combination of a fluorescent domain which can spontaneously photoswitch, with a sensing domain which responds to a desired input (e.g., such as the concentration or availability of a molecule of interest).

As used herein, the term “photoswitch” refers to cis-trans isomerization or protonation of a fluorophore.

As used herein, the term “pH-Countdown” is a Countdown sensor that can be used to detect pH.

It must be noted that, as used in the specification and the appended claims, the singular forms “a,” “an” and “the” include plural referents unless the context clearly dictates otherwise.

### Countdown Sensors

Described herein is a class of biosensors, spontaneous isomerization rate sensors, or “Countdown sensors” that combine the ease-of-use of an intensity biosensor with the unambiguous quantitation of a lifetime biosensor. Also described herein are novel engineered fluorescent domains (“countdown domains”) which are especially well suited to be used as the fluorescent domain of a countdown sensor.

In certain aspects, described herein is a protein biosensor, comprising: a fluorescent domain; and an analyte binding domain; wherein the fluorescent domain can spontaneously photoswitch by cis-trans isomerization or protonation; and wherein the rate of isomerization or rate of protonation is altered by binding of the analyte binding domain to an analyte of interest.

In certain embodiments, the photoswitching changes fluorescent intensity or the fluorescent color of the fluorescent domain. In certain embodiments, the analyte binding domain is attached to the N-terminus of the fluorescent domain. In certain embodiments, the analyte binding domain is attached to the C-terminus of the fluorescent domain.

The fluorescent domain can be any suitable fluorophore that can spontaneously “photoswitch”: they change shape (e.g., via cis-trans isomerization or protonation) between two or more states with different fluorescent properties (e.g. fluorescent intensity or fluorescent color). In certain embodiments, the fluorescent domain comprises the green fluorescent protein (GFP) or rsCherry.

The analyte of interest can be any molecule of interest, whether it be a small molecule, an ion, or a larger protein or complex cellular structure. In certain embodiments, the analyte of interest is hydrogen ion.

In certain aspects, the “Countdown sensor” disclosed herein comprises the combination of a fluorescent domain which can spontaneously photoswitch, with a sensing domain which responds to the desired input (e.g. hydrogen ion concentration). The countdown sensor is “read” by measuring the photoswitching rate, and, for example, comparing the observed rate to a calibration curve of rate vs. quantity-to-be-sensed (e.g., hydrogen ion concentration). The decay of fluorescent intensity vs. time (due to spontaneous photoswitching of different fluorescent domains) can be made to depend on the concentration of different small molecules (in this case, hydrogen ion).

Since spontaneous photoswitching rate does not depend on the concentration of the countdown sensor, or the measurement instrument, such measurements can be quantitative like a lifetime sensor. Since spontaneous photoswitching rates can be measured with a series of fluorescence intensity measurements, such measurements can be fast, cheap, and easy to produce like an intensity sensor.

Unfortunately, the vast majority of fluorophores which spontaneously photoswitch do so extremely slowly (spontaneous photoswitching half-lives of hours or even days). Disclosed herein are a novel family engineered green fluorescent proteins which spontaneously photoswitch with half-lives ranging from hours to seconds. Their photoswitching rates become faster or slower depending on perturbations applied to the two “tails” of the protein (their C and N termini). For example, the amino acid sequence set forth in SEQ ID NO: 1 encodes one of these engineered rapidly spontaneously photoswitching green fluorescent proteins.

All photoswitchable fluorescent proteins “relax”, spontaneously equilibrating to some steady-state ratio of active and inactive states. Unfortunately, this process typically takes hours—much too slow for most sensing applications. In certain embodiments, the photoswitchable fluorescent proteins (also termed “Countdown”) disclosed herein rapidly spontaneously equilibrate to the inactive state, in seconds instead of hours. In certain embodiments, Countdown photoswitches between a bright green fluorescent active state and a nonfluorescent inactive state. In certain embodiments, Cyan light activates and excites Countdown, violet light inactivates Countdown, and thermal equilibrium favors the inactive state.

This disclosure includes many other examples of spontaneous isomerization rate sensors and spontaneously phostoswitching fluorescent proteins, which can vary in their spontaneous photoswitching half-life, their photoswitching rate, their cross-section for light-driven on-switching, their cross-section for light-driven off-switching, their equilibrium degree of activation, the brightness/intensity of their “on” state, the brightness/intensity of their “off” state, their maturation time, and their tendency to oligomerize. Informally, the spontaneous photoswitching rate of the protein “countdown” sensor depends on how you tug on its tails (i.e., put strain on the N and/or C termini of the fluorescent domain). This is also true of other rapidly spontaneously photoswitching fluorescent proteins, for example, rsCherry.

In certain embodiments, discloses herein are Countdown sensors with a range of photoswitching and spontaneous decay rates. In certain embodiments, the Countdown sensor has a photoswitching rate that is approximately the same as the photoswitching rate of the Countdown sensor encoded by the amino acid sequence set forth in SEQ ID NO. 1.

In certain embodiments, the Countdown sensor has a photoswitching rate that is approximately 1-2, 1-10, 1-5, 1-1.1, 1.1-1.2-1.2-1.3, 1.3-1.4, 1.4-1.5, 1.5-1.6, 1.6-1.7, 1.7-1.8, 1.8-1.9, 1.9-2, 2-2.5, 2.5-3, 3-3.5, 3.5-4, 4-4.5, 5-5.5, 5.5-6, 6-6.5, 6.5-7, 7-7.5, 7.5-8, 8-8.5, 8.5-9, 9-9.5, 9.5-10, 1-100, 10-15, 15-20, 20-30, 30-40, 40-50, 50-100, 100-1,000, 100-200, 200-300, 300-400, 400-500, 500-1,000, 1,000-10,000 fold less than the photoswitching rate of the biosensor encoded by the amino acid sequence set forth in SEQ ID NO. 1. In certain embodiments, the Countdown sensor has a photoswitching rate that is approximately 1-1.1, 1.1-1.2-1.2-1.3, 1.3-1.4, 1.4-1.5, 1.5-1.6, 1.6-1.7, 1.7-1.8, 1.8-1.9, 1.9-2, 2-2.5, 2.5-3, 3-3.5, 3.5-4, 4-4.5, 5-5.5, 5.5-6, 6-6.5, 6.5-7, 7-7.5, 7.5-8, 8-8.5, 8.5-9, 9-9.5, 9.5-10, 10-15, 15-20, 20-30, 30-40, 40-50, 50-100, 100-1,000, 100-200, 200-300, 300-400, 400-500, 500-1,000, 1,000-10,000 fold greater than the photoswitching rate of the biosensor encoded by the amino acid sequence set forth in SEQ ID NO. 1.

In certain embodiments, the Countdown sensor comprises a protein encoded by an amino acid sequence set forth in SEQ ID NO. 1. In certain embodiments, the Countdown sensor comprises a protein encoded by amino acid sequence that is greater than 50%, 60%, 70%, 80%, 85%, 90%, 91%, 92%, 93%, 94%, 95%, 96%, 97%, 98%, 98.5%, 99%, 99.1%, 99.2%, 99.3%, 99.4%, 99.5%, 99.6%, 99.7%, 99.8%, 99.9% identical to the amino acid sequence set forth in SEQ ID NO. 1.

In certain embodiments, the Countdown sensor is a fusion protein and comprises one or more additional proteins.

### Methods of Engineering Countdown Sensors

Also disclosed herein are methods of engineering Countdown sensors. Anything that can be made to “tug” on the “tails” of a rapidly spontaneously photoswitching fluorescent protein can be measured via the same optical measurement. There are many ways to use the readout of the “tugging” property of a fluorophore to construct a biosensor. For example, a countdown sensor can be attached to a hydrogen ion binding domain to produce a green pH countdown sensor. In another example, rsCherry can be attached to a hydrogen ion binding domain to produce a red pH countdown sensor.

Construction of novel biosensors including the examples disclosed herein are engineered and constructed rapidly due to the remarkable modularity of countdown sensors. By simply attaching selected binding domains for an analyte of interest to the native C and N termini of a spontaneously photoswitching fluorescent domain, the fluorescent properties are preserved to an exceptional degree, primarily modulating their photoswitching rates with minimal effects on other properties like brightness.

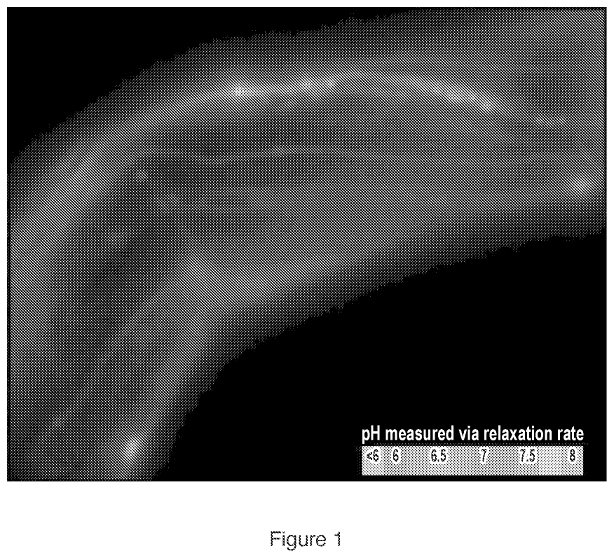

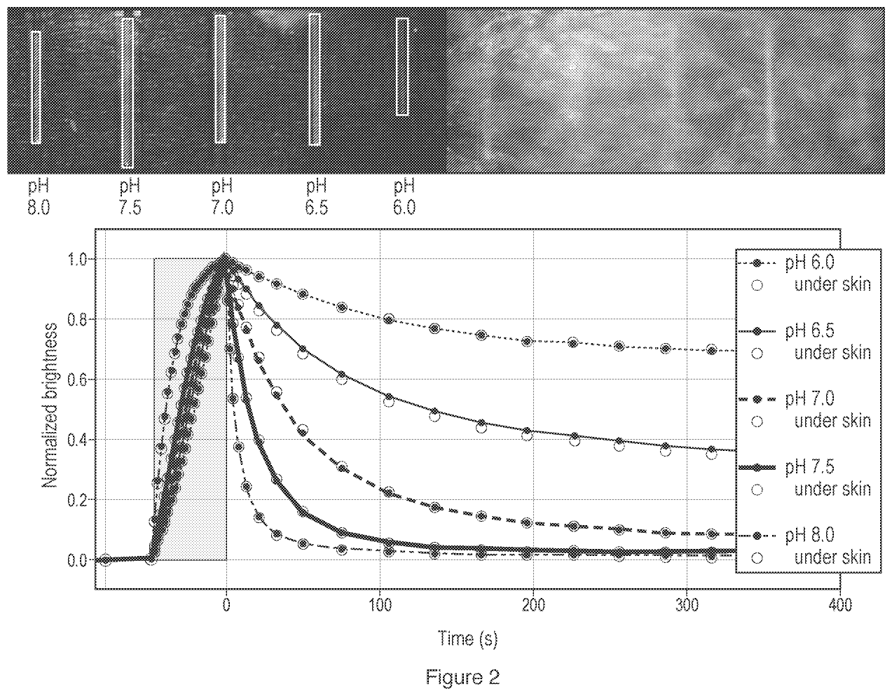

pH-Countdown's apparent decay rate depends weakly on the amount of illumination during measurement, because cyan light drives both activation and excitation. This can be calibrated by using the same illumination for calibration and measurement (FIGS. 1 and 3-5), or predicted since the activation rate reveals the amount of illumination, or eliminated by using low intensity illumination (FIGS. 2 and 8). A relaxation sensor engineered from a photoswitcher with decoupled activation/excitation would also eliminate this concern (Brakemann 2011). The relaxation of pH-Countdown also takes at least a few seconds; signal interpretation is much simpler if the sample holds still during this time. Engineering faster relaxation sensors or developing more sophisticated analysis would allow faster-moving samples.

In addition, described herein are methods to modularly combine appropriate fluorescent domains with appropriate sensing domains in order to rapidly, easily produce novel countdown sensors.

### Methods of Use

Disclosed herein are methods of using the Countdown sensors of this disclosure to identifying the concentration of an analyte of interest in a sample. In certain embodiments, disclosed herein is a method of identifying the concentration of an analyte of interest in a sample, comprising contacting the sample with a protein biosensor of the instant disclosure. In certain embodiments of the methods, a change in fluorescent intensity of the biosensor is correlated with the concentration of the analyte of interest in the sample. In certain embodiments, the change of fluorescent intensity of the biosensor is not dependent on the concentration of the biosensor.

The Countdown sensors described herein can be used for detecting analytes of interest in vitro or in vivo. The Countdown sensors described herein can be used for detecting either small molecules/analytes (e.g., H⁺), or larger molecules via coupling to a binding domain. Any process that can be made to “tug” on the “tails” of a rapidly spontaneously photoswitching fluorescent protein can be measured via the same optical measurement. Thus, any analyte can be measured as long as it is coupled to the Countdown sensor to modulate the photoswitching rate. In a particular non-limiting example, Countdown sensors could be used to measure cellular physiological phenomenon, such as, but not limited to, being used as a tension sensor for actin cytoskeletal dynamics by coupling it to components of the cytoskeleton.

In certain embodiments, the sensors described herein allow for sensing an analyte using a Countdown sensor described herein and using the same spectral channel of the fluorescent domain of the Countdown sensor to detect another molecule or analyte of interest. In certain embodiments, a plurality of the Countdown sensors described herein are used in combination for performing multiplexed fluorescent imaging assays in a single spectral channel or more than one spectral channel.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: pH Biosensors In Vivo

C. elegans worms were injected with a pH countdown sensor described herein (SEQ ID NO 1) and pRF4 (a selection marker) to express pH-countdown from an extrachromosomal array. The worms were imaged on a standard widefield microscope, and the decay rates were converted to pH using a calibration curve at the same temperature. See FIG. 1. The pH converted image (FIG. 1) shows that most tissues display pH values in the neutral physiological range, with the exception of the punctate structures, which can be neutral or acidic.

### Example 2: pH Biosensors Quantitatively Measure pH Despite Autofluorescent Background and Attenuation

As a proof-of-principle, a Countdown was used to make a relaxation sensor for pH. Three rounds of mutagenesis was performed, screening for strong pH-dependence in the first several seconds of the relaxation curve, to produce “pH-Countdown”. FIG. 2 shows photoactivation/relaxation curves for pH-Countdown at 25° C., as a function of pH, acquired with a simple LED-based fluorescence photography system.

FIG. 2 depicts data using the pH-Countdown biosensor, a photoswitchable fluorescent protein-based relaxation sensor which quantitatively measures pH despite autofluorescent background and attenuation due to absorption and scattering. Upper left panel: Timelapse photography of fluorescence (green) and reflected light (grayscale) during photoactivation and relaxation of pH-Countdown, in five capillary tubes, each buffered at a different pH. For scale, the capillary inner diameter is 580 m. Lower panel: Normalized fluorescence signal vs. time for each of the capillary tubes shown in the upper panels. Filled circles are from the highlighted regions in the upper left panel, open circles are from the upper right panel. Cyan box shows photoactivation interval (488 nm, 10 mW/cm²). Upper right panel: Like the upper left panel, except the capillary tubes are underneath ˜1 mm of chicken skin.

As expected, pH-Countdown's relaxation curves vary dramatically with pH, over a large pH range (bottom panel). After only a few seconds, the amount of relaxation for each pH is clearly distinguishable, allowing reasonably fast measurement (0.1-1 Hz). The short illumination duration during each image (500 s, 10 mW/cm2) does not cause measurable photoactivation, and the low resolution yields a population-averaged measurement rather than stochastic single-molecule kinetics. Note that relaxation rates are several-fold faster at 37° C. than 25° C., and this sensor generally requires temperature calibration to yield quantitative pH measurements.

The key point of FIG. 2 is that burying our sample underneath a millimeter of partially opaque, highly autofluorescent chicken skin (upper right panel) does not affect the apparent pH reported by our relaxation sensor (bottom panel, open vs. filled circles). This is remarkable; no other fluorescent sensor has this property.

### Example 3: Relaxation Sensing In Vivo with pH-Countdown

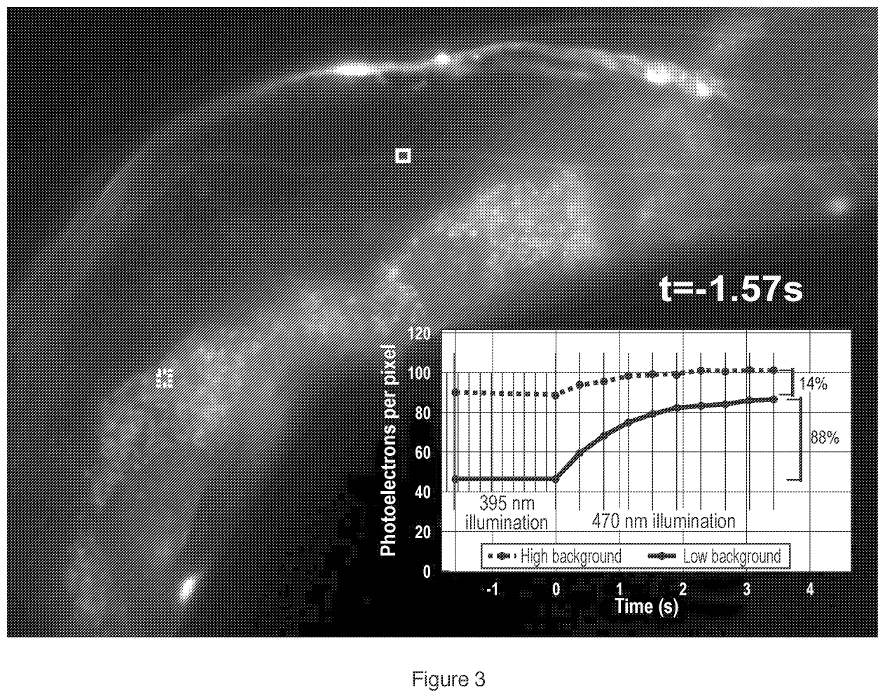

To demonstrate relaxation sensing in living organisms, we transfected yeast (S. cerevisiae), mammalian cells (COS-7), and worms (C. elegans) with pH-Countdown. Due to high autofluorescence in the GFP channel from gut granules (Teuscher 2018), C. elegans is the most challenging, and therefore the most interesting sample. FIG. 3 shows pH-Countdown photoactivation in a live (but paralyzed) C. elegans.

The GFP-channel time lapse in FIG. 3 shows both autofluorescent background and pH-Countdown signal. We first drive pH-Countdown toward the inactive state with violet light (inset, vertical violet lines), and then acquire a series of images with cyan light (inset, vertical cyan lines) which drives pH-Countdown into the active state (and out of thermal equilibrium).

In some regions (solid green box), the signal is mostly due to the sensor, but many regions (dotted magenta box) have sensor-to-background ratios substantially below 1: most of the photoelectrons are from autofluorescent background. This situation is typical in C. elegans. No intensity- or lifetime*-based sensor tested provides a way to distinguish signal photoelectrons from background, leading to systematically distorted results in C. elegans.

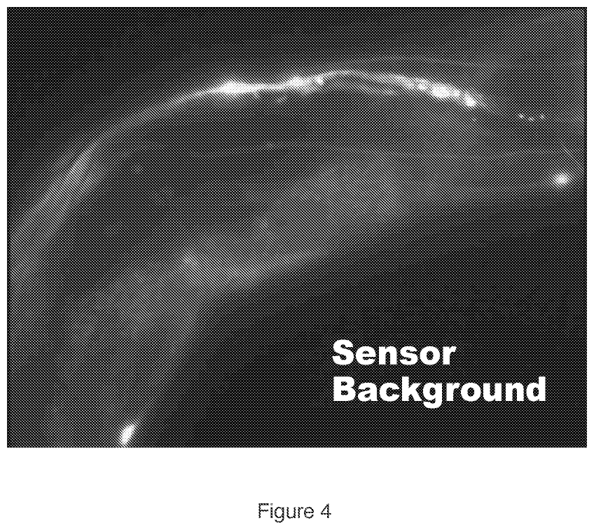

FIG. 3 shows that the signal produced by pH-Countdown is distinguishable from autofluorescence, because pH-Countdown gets brighter with each subsequent image, and autofluorescence does not. As shown in FIG. 4, relaxation sensing inherently rejects background, even in regions with terrible signal-to-background ratio.

The green “sensor” overlay in FIG. 4 is simply the difference between the most-photoactivated and least-photoactivated frames from the time lapse in FIG. 3. The paralyzed worm does not move during this ˜3.5 s interval, and neither pH-Countdown nor autofluorescence bleach measurably due to the 10 cyan exposures used for a single photoactivation. Therefore, the green “sensor” image is due only to photoactivation of pH-Countdown, and Poisson fluctuations from both the sensor and the background. Poisson fluctuations have zero mean, so background causes noise in the sensor signal, but no systematic distortion. Poisson fluctuations scale like the square root of the total counts, so even though the signal-to-background ratio is <1 at many pixels, the signal-to-noise ratio is >1 for most pixels with measurable sensor.

To estimate pH, only the green “sensor” channel is needed. However, a “background” signal can also be estimated, which is not due to pH-Countdown, shown as a magenta overlay in FIG. 4. In this sample, the background channel clearly shows autofluorescent gut granules, familiar to any microscopist who has studied C. elegans with fluorescence. Since GFP-channel autofluorescence is quite similar to GFP fluorescence, this implies an exciting possibility: sensing with pH-Countdown uses the GFP spectral channel, but does not interfere with using GFP to tag another structure. Relaxation sensors like pH-Countdown are single-spectral-channel sensors, but from a multiplexing perspective, they consume zero spectral channels. During mutagenesis to produce pH-Countdown, an entire mutational series was produced with intermediate photoswitching and spontaneous decay rates, and we expect that these mutants could be the basis for highly-multiplexed fluorescent imaging in a single spectral channel.

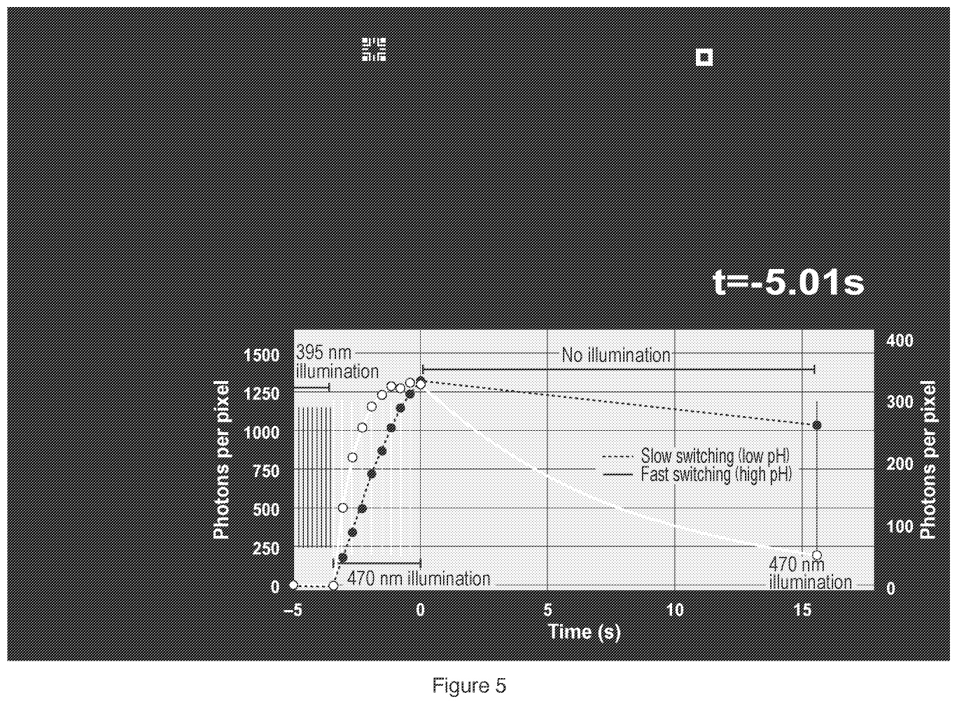

FIGS. 3 and 4 show that the amount of photoswitching lets us separate signal from background. FIG. 5 shows how the rate of (photo)switching reveals our quantity of interest: pH.

The time lapse in FIG. 5 shows the data from FIG. 3, with background subtracted as in the green overlay of FIG. 4. In addition to photoactivation, we also now show the relaxation interval, as illustrated in FIG. 2. Unlike FIG. 2, we must leave the illumination off during relaxation to avoid perturbing the apparent relaxation rate. Distinct structures in this time lapse photoactivate and relax at clearly distinguishable rates (e.g. dotted blue box vs. solid yellow box).

As shown in FIG. 2, the (photo)switching rates of pH-Countdown depend strongly on pH. We know pH-Countdown switching rates also depend (more weakly) on temperature, but as far as we know, there is no evidence for wild multi-° C./μm thermal gradients in live C. elegans. We are also unable to identify any other substantial off-target sensing for pH-Countdown, although we note that such testing is never exhaustive.

Therefore, we choose to interpret the photoactivation and relaxation rates shown in FIG. 5 as a map of pH vs. position, which we plot in false color in FIG. 1.

We define the “relaxation ratio” as a pixelwise ratio of differences:

$$\frac{( Image after activation ) - ( Image after relaxation )}{( Image after activation ) - ( Image after deactivation )}$$

Where “image after deactivation”, and “image after activation” and “image after relaxation” correspond to frames 1, 10, and 11 of FIG. 5, respectively. The relaxation ratio is a simple, robust estimator. It depends strongly on the rate of relaxation, but not on the quantity of sensor or quantity of background, and it depends only weakly on the amount of illumination. By comparing the observed relaxation ratio to a calibration dataset, we can account for the effects of illumination intensity, and convert our map of relaxation ratios to the map of pH vs. position shown in FIG. 1.

It's inherently futile to validate a sensor via an in vivo measurement, but the pH map in FIG. 1 has several reassuring features. The inferred pH values are broadly consistent with the range we expect in living creatures. Continuous structures have smoothly-varying pH values, suggesting our measured values are not dominated by noise. Distinct structures have distinct pH values, suggesting our measured values are not independent of the sample. Some of the bright punctate structures have especially low pH, but some also have fairly high pH, suggesting the results are not simply an aggregation artifact. The results are highly encouraging, and consistent with all previous experience using pH-Countdown.

There is a tradeoff between speed and signal-to-noise ratio (SNR). FIG. 1 shows measured pH maps for three different values of Relaxation Interval, and the longest interval shows superior SNR compared to the shortest interval. In this case, waiting longer improves SNR, but this is only because the sample holds still.

### Example 4: Relaxation Sensing Using pH-Countdown in Mice

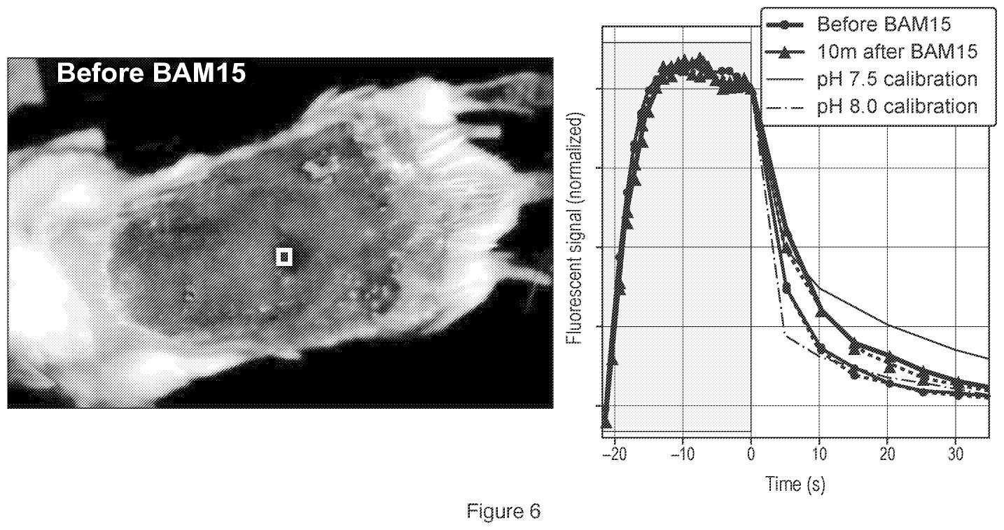

FIG. 6 shows pH-Countdown measurements in a live (but anaesthetized) Mus musculus (mice). BAM15 is a “mitochondrial uncoupler” (Kenwood 2014), which has been explored as a potential anti-obesity drug (Alexopoulos 2020). Mitochondrial uncouplers transport protons into the mitochondrial matrix, which we would expect to lower the mitochondrial pH. By expressing pH-Countdown in the mitochondria of a tumor in a living mouse, we can directly quantitatively interrogate the proposed mechanism of action of this drug.

As shown in FIG. 6, the signal from fully subcutaneous mito-pH-Countdown clearly reveals a drug-induced shift in mitochondrial pH, of roughly 0.2 pH units. This agrees qualitatively with similar measurements in cultured cells, where mitochondrial uncouplers rapidly lower mitochondrial pH. However, it disagrees quantitatively with our results when the same cell type is given the same drug in a dish, which yields a shift closer to 1 pH unit. It is not surprising that an animal buffers the effects of a drug on its cells; however, this approach allows for measurement of the difference quantitatively. In addition, pharmacokinetics are explored; for example, the mitochondrial pH begins to return to baseline in an interval consistent with BAM15's reported half-life.

### Informal Sequence Listing

SEQ ID NO. 1
MVSKGEENNMAVIKPDMKIKLRMEGSVNGHRFRIEGVGLGKPLEGKQSMD
LKVKEGGPLPFAYDILTMAFCYGNRVFAKYPENIVDYFKQSFPEGYSWER
QMIYEDGGICVATNDITLDGDCMISEIRFKGVNFPANGPVFQKRTVKWEL
SHEKLYARDGLLYSDGNYALSLEGGGHYRCDNKTTYKAKKVVQLPDYHWV
THSIVIKSHDKDYSNVNLHEHAEAHSELPRQAMDELYK

1. A protein biosensor, comprising:
a fluorescent domain; and
an analyte binding domain; wherein
the fluorescent domain can spontaneously photoswitch by cis-trans isomerization or protonation; and wherein
the rate of isomerization or rate of protonation is altered by binding of the analyte binding domain to an analyte of interest.

2. The protein biosensor of claim 1, wherein the photoswitching changes fluorescent intensity or the fluorescent color of the fluorescent domain.

3. The protein biosensor of claim 1 or 2, wherein the analyte binding domain is attached to the N-terminus of the fluorescent domain.

4. The protein biosensor of claim 1 or 2, wherein the analyte binding domain is attached to the C-terminus of the fluorescent domain.

5. The protein biosensor of any one of claims 1-4, wherein the fluorescent domain is green fluorescent protein (GFP) or rsCherry.

6. The protein biosensor of any one of the above claims, wherein the analyte of interest is hydrogen ion.

7. A method of making a protein biosensor, comprising:
attaching an analyte binding domain to a fluorescent domain; wherein
the fluorescent domain can spontaneously photoswitch by cis-trans isomerization or protonation; and wherein
the rate of isomerization or rate of protonation is altered by binding of the analyte binding domain to an analyte of interest.

8. A method of identifying the concentration of an analyte of interest in a sample, comprising contacting the sample with a protein biosensor of any one of the above claims.

9. The method of claim 8, wherein
a change in fluorescent intensity of the biosensor is correlated with the concentration of the analyte of interest in the sample.

10. The method of claim 8 or 9, wherein
the change of fluorescent intensity of the biosensor is not dependent on the concentration of the biosensor.

11. The protein biosensor of any one of the above claims, wherein the protein biosensor comprises SEQ ID NO. 1.

12. A kit comprising the protein biosensor of any one of the above claims and instructions for use.


SEQUENCE LISTING (1 sequence)

SEQ ID NO: 1 - protein, 238 aa, synthetic construct
MVSKGEENNMAVIKPDMKIKLRMEGSVNGHRFRIEGVGLGKPLEGKQSMDLKVKEGGPLPFAYDILTMAFCYGNRVFAKYPENIVDYFKQSFPEGYSWERQMIYEDGGICVATNDITLDGDCMISEIRFKGVNFPANGPVFQKRTVKWELSHEKLYARDGLLYSDGNYALSLEGGGHYRCDNKTTYKAKKVVQLPDYHWVTHSIVIKSHDKDYSNVNLHEHAEAHSELPRQAMDELYK